(19) United States
(12) Patent Application Publication
(10) Pub. No.: US 2009/0018110 A1
(43) Pub. Date: Jan. 15, 2009

(54) Haptoglobin genotyping for prognosis and treatment of chronic vasospasm following subarachnoid hemorrhage (SAH)

(76) Inventors: Andrew Levy, Haifa (IL); Noah Berkowitz, New Rochelle, NY (US)

Correspondence Address: Pearl Cohen Zedek Latzer, LLP, 1500 Broadway, 12th Floor, New York, NY, 10036, US

(21) Appl. No.: 12/155,560

(22) Filed: Jun. 5, 2008

Related U.S. Application Data
(63) Continuation of application No. 60/924,936, filed on Jun. 6, 2007.
(60) Provisional application No. 60/924,935, filed on Jun. 6, 2007.

(51) Int. Cl. A61K 31/555; G01N 33/53; A61P 9/14; C12M 1/34

(52) U.S. Cl. 514/186; 435/7.1; 435/7.92; 435/287.2; 514/184

(57) ABSTRACT

This invention relates to methods and systems for providing a prognosis to a subject on developing vasospasm as a results of hemorrhagic event, and compounds and compositions for treatment thereof. Specifically, the invention relates to the use of haptoglobin genotyping in the prognosis of the development of vasospasm following SAH, and antioxidants such as glutathione peroxidase mimetics for treatment.

### CROSS-REFERENCE TO RELATED APPLICATIONS

This application claims priority under 35 § 119(e) to provisional application Ser. Nos. 60/924,936 and 60/924,935, both filed Jun. 6, 2007, and both of which are incorporated herein by reference in their entireties.

### FIELD OF INVENTION

This invention is directed to methods and systems for providing a prognosis for, and methods and compositions for treatment of, a subject of developing vasospasm as a result of subarachnoid hemorrhage (SAH). Specifically, the invention is directed to the use of haptoglobin genotyping in the prognosis of the development of vasospasm resulting from SAH, and to antioxidant therapies therefor.

### BACKGROUND OF THE INVENTION

Cerebral arterial vasospasm is the leading cause of morbidity and mortality following aneurysmal subarachnoid hemorrhage (SAH). In humans, cerebral vasospasm is a biphasic phenomenon, where acute vasospasm occurs within hours of the hemorrhage and is followed by a delayed, sustained narrowing of the cerebral arteries four to 21 days later. This delayed narrowing leads to delayed ischemic neurological deficits, which result in permanent deficits and even death in 20 to 40% of patients.

There is growing evidence that inflammation and, more specifically, leukocyte-endothelial cell interactions are the root cause of vasospasm after SAH. Clinical evidence supporting the role of inflammation includes increased body temperature, elevated white blood cell counts, and increased levels of pro-inflammatory adhesion molecules, namely intercellular adhesion molecule-1 (ICAM-1, CD54), in patients who develop vasospasm. Furthermore, inhibition of leukocyte-endothelial cell interactions decreases the incidence of vasospasm in experimental models. Despite these findings, the ability to predict which patients will develop vasospasm following SAH is limited. Prospective identification of patients at greater risk for developing vasospasm would allow for selective early administration of potentially helpful therapies. It may also lead to novel therapeutic approaches aimed at reducing the incidence and severity of vasospasm following SAH.

Subarachnoid hemorrhage may be caused by trauma or by non-traumatic incidents, such as a ruptured intracranial aneurysm, arteriovenous malformation, or vasculitis. As mentioned above, cerebral arterial vasospasm is the leading cause of morbidity and mortality in patients surviving subarachnoid hemorrhage (SAH). Oxyhemoglobin appears to be responsible for vasospasm after SAH. Much of the existing evidence suggests that oxyhemoglobin is the clinically-relevant vasospastic agent. The presence of high concentrations of extracellular oxyhemoglobin in the cerebrospinal fluid (CSF) correlates with the presence of spasm. Concentrations of extracellular oxyhemoglobin are highest 4 to 7 days post-SAH, correlating with the onset of vasospasm. Furthermore, intact erythrocytes are inert, whereas lysed erythrocytes are vasospastic.

Release of blood into the subarachnoid space occurs following breach of a blood vessel, as in the rupture of an aneurysm in the arterial blood supply to the brain. The pooling of blood in the subarachnoid space exposes the brain dura mater to blood contact and over a period of two to three days a number of events occur: first, red blood cells (RBC) begin to lyse, liberating RBC components including free hemoglobin into the surrounding subarachnoid space, and the subsequent progressive conversion of oxyhemoglobin to methemoglobin with the possible production of superoxide anion radicals and other reactive oxygen species. In addition, inflammation and, more specifically, leukocyte-endothelial cell interactions are the root cause of vasospasm. Finally, RBC components mediate the inflammation underlying the pathogenesis of cerebral vasospasm.

Cerebral vasospasm is delayed onset cerebral artery narrowing in response to blood clots left in the subaracbnoid space after spontaneous aneurysmal subarachnoid hemorrhage (SAH). It is angiographically characterized as the persistent luminal narrowing of the major extraparenchymal cerebral arteries and affects the cerebral microcirculation and causes decreased cerebral blood flow (CBF) and delayed ischemic neurological deficits. A number of studies have demonstrated morphological changes in cerebral arteries after SAH. The impaired dilator and increased constrictor mechanisms that occur after SAH may be caused by oxyhaemoglobin produced by erythrocytes that inactivates NO in the subarachnoid space. Alternatively it may be due to an impaired activity of soluble guanylate cyclase resulting in reduced basal levels of cGMP in cerebral vessels and so a reduced responsiveness to NO.

Reperfusion following resolution vasospasm resulting from traumatic head injury, leads to additional neurological injury such as phagocytic damage to the endothelium and surrounding tissues and the release of oxygen-derived free radicals (reactive oxygen species or ROS). ROS creates damage to vascular, neuronal, and glial membranes, with excitotoxic, intracellular calcium overload depletion of nitric oxide (NO) through lipid peroxidation reactions and excitatory amino acid release, glutamate, overwhelming the antioxidant enzyme mechanisms of the affected cells.

Due to the mortality and morbidity associated with vasospasm resulting from hemorrhagic events there continues to exist a need for effective treatments.

### SUMMARY OF THE INVENTION

In one embodiment, the invention provides a method of providing a prognosis for development of vasospasm in a subject, comprising the steps of: obtaining a biological sample from a subject following a hemorrhagic event; determining the haptoglobin (Hp) genotype in the biological sample, whereby a subject expressing a Hp 2-2 genotype has a high risk of developing vasospasm; and providing the prognosis based on the subject's haptoglobin genotype.

In another embodiment, the invention provides a system for providing a prognosis for development of vasospasm in a subject, comprising: a reagent, a packaging material; and instructions for determining the subject's Haptoglobin genotype.

In another embodiment, the invention provides a method of treating a vasospasm in a subject, comprising: contacting said subject, wherein the subject has suffered a hemorrhagic event with an effective amount of a composition comprising an antioxidant or its isomer, metabolite, and/or salt therefore, thereby treating vasospasm.

In another embodiment, the invention provides a method of inhibiting or suppressing a vasospasm, comprising: contacting said subject, wherein the subject has suffered a hemorrhagic event with an effective amount of a composition comprising an antioxidant or its isomer, metabolite, and/or salt therefore, thereby inhibiting or suppressing vasospasm.

In one embodiment, the invention provides a method of reducing symptoms associated with a vasospasm in a subject, comprising: contacting said subject, wherein the subject has suffered a hemorrhagic event with an effective amount of a composition comprising an antioxidant or its isomer, metabolite, and/or salt therefore, thereby reducing symptoms associated with vasospasm.

In another embodiment, the invention provides a method of treating a vasospasm, inhibiting or suppressing a vasospasm, or reducing symptoms of a vasospasm in a subject, comprising: obtaining a biological sample from a subject following a hemorrhagic event; determining the haptoglobin (Hp) genotype in the biological sample, and for subjects with a Hp 2-2 genotype, contacting said subject with an effective amount of a composition comprising an antioxidant or its isomer, metabolite, and/or salt therefore, thereby reducing symptoms associated with vasospasm.

In another embodiment, the invention provides a composition for treating a vasospasm, inhibiting or suppressing a vasospasm, or reducing symptoms of a vasospasm in a subject wherein the subject suffered a hemorrhagic event, comprising: a therapeutically effective amount of a composition comprising an antioxidant or its isomer, metabolite, and/or salt therefore.

In another embodiment, the invention provides a composition for treating a vasospasm, inhibiting or suppressing a vasospasm, or reducing symptoms of a vasospasm in a subject wherein the subject suffered a hemorrhagic event, comprising: a therapeutically effective amount of a composition comprising a glutathione peroxidase mimetic or its isomer, metabolite, and/or salt therefore.

In another embodiment, the invention provides a method of treating a vasospasm, inhibiting or suppressing a vasospasm, or reducing symptoms of a vasospasm in a subject, comprising: obtaining a biological sample from a subject following a hemorrhagic event; determining the haptoglobin (Hp) genotype in the biological sample, and for subjects with a Hp 2-2 genotype, contacting said subject with an effective amount of a composition comprising a glutathione peroxidase mimetic or its isomer, metabolite, and/or salt therefore, thereby reducing symptoms associated with vasospasm.

Other features and advantages of the present invention will become apparent from the following detailed description examples and figures. It should be understood, however, that the detailed description and the specific examples while indicating preferred embodiments of within the spirit and scope of the invention will become apparent to those skilled in the art from this detailed description.

### BRIEF DESCRIPTION OF THE DRAWINGS

The invention will be better understood from a reading of the following detailed description taken in conjunction with the figures and examples in which like reference designators are used to designate like elements or findings, and in which:

FIG. 1 shows the percent lumen patency of the basilar artery was determined 24 hours following SAH. The lumen patency was significantly decreased in Hp 1-1 blood-injected mice as compared to Hp 1-1 saline-injected mice, *p=0.01 (Student-Newman-Keuls). Likewise, the lumen patency was significantly decreased in Hp 2-2 blood-injected mice as compared to Hp 2-2 saline-injected mice, **p<0.001 (Student-Newman-Keuls). More importantly, the lumen patency was significantly decreased in Hp 2-2 blood-injected mice as compared to Hp 1-1 blood-injected mice, p<0.001 (Student-Newman-Keuls). Values are the mean±SEM of 10 mice per group;

FIG. 2 shows the activity level, as described in Table 1, was assessed 24 hours following SAH. The activity level was not significantly decreased in Hp 1-1 blood-injected mice as compared to Hp 1-1 saline-injected mice, p=0.08 (Student-Newman-Keuls). The activity level, however, was significantly decreased in Hp 2-2 blood-injected mice as compared to Hp 2-2 saline-injected mice, **p<0.001 (Student-Newman-Keuls). More importantly, the activity level was significantly decreased in Hp 2-2 blood-injected mice as compared to Hp 1-1 blood-injected mice, p<0.001 (Student-Newman-Keuls). Values are the mean±SEM of 10 mice per group;

FIG. 3 shows immunohistochemical analysis of mouse basilar artery sections demonstrate that (d) blood-injected Hp 2-2 mice have more extensive macrophage/neutrophil infiltration into the subarachnoid space than (b) blood-injected Hp 1-1 mice (d). (a) Saline-injected Hp 1-1 mouse. (b) Blood-injected Hp 1-1 mouse. (c) Saline-injected Hp 2-2 mouse. (d) Blood-injected Hp 2-2 mouse. Scale bar, 50 μm; and

FIG. 4 shows the number of macrophages/neutrophils per basilar artery section was determined 24 hours following SAH. The number of macrophages/neutrophils was significantly increased in Hp 1-1 blood-injected mice as compared to Hp 1-1 saline-injected mice, *p=0.004 (Student-Newman-Keuls). Likewise, the number of macrophages/neutrophils was significantly increased in Hp 2-2 blood-injected mice as compared to Hp 2-2 saline-injected mice, **p=0.002 (Student-Newman-Keuls). More importantly, the number of macrophages/neutrophils was significantly increased in Hp 2-2 blood-injected mice as compared to Hp 1-1 blood-injected mice, p=0.009 (Student-Newman-Keuls). Values are the mean±SEM of 5 mice per group.

### DETAILED DESCRIPTION OF THE INVENTION

This invention relates in one embodiment to methods and systems for providing a prognosis to a subject on developing vasospasm as a results of subarachnoid hemorrhage (SAH). In another embodiment, the invention provides for the use of Haptoglobin genotyping in the prognosis of the development of vasospasm resulting from SAH. In other embodiment the invention relates to compositions and methods for treating vasospasm. In another embodiment, the invention is provides methods and compositions for treating vasospasm as a result of a hemorrhagic event comprising contacting the subject with a composition comprising an antioxidant such as a glutathione peroxidase mimetic.

SAH after aneurysmal rupture occurs in 10.5 cases per 100,000 individuals per year, which equates to approximately 30,000 cases annually in the United States. The most serious complication following aneurysmal SAH is chronic cerebral vasospasm. In one embodiment, the term “Chronic cerebral vasospasm” refers to the delayed and sustained narrowing of the cerebral arteries that occurs four to 21 days after a SAH. In another embodiment, as a result of SAH, patients fall into one of three categories: (1) approximately 30% of patients develop angiographic vasospasm and also clinical symptoms of ischemia; (2) 50% of patients develop angiographic vasospasm, but no clinical symptoms; and (3) 20% have neither angiographic nor clinical evidence of vasospasm. In one embodiment, vasospasm also occur as result of traumatic brain injury, or in another embodiment, as the result of craniotomy for tumors, or in another embodiment, as the result of meningitis. In one embodiment, the methods and systems described herein, have the ability to provide prognosis to a subject on developing vasospasm as a results of traumatic brain injury, craniotomy for tumors, meningitis or their combination.

In one embodiment, release of blood into the subarachnoid space occurs following breach of a blood vessel, as in traumatic brain injury in one embodiment. In another embodiment, the pooling of blood in the subarachnoid space exposes the brain dura mater to blood contact. In certain embodiments, over a period of two to three days a number of events occur: in one embodiment, red blood cells (RBC) begin to lyse, liberating RBC components including free hemoglobin into the surrounding subarachnoid space, and the subsequent progressive conversion of oxyhemoglobin to methemoglobin with the possible production of superoxide anion radicals and other reactive oxygen species. In one embodiment, inflammation and, more specifically, leukocyte-endothelial cell interactions are the root cause of vasospasm. In another embodiment, RBC components mediate the inflammation underlying the pathogenesis of cerebral vasospasm.

In one embodiment, patients who are febrile following SAH have worse outcomes than patients who were euthermic. In another embodiment, patients with symptomatic vasospasm have a sustained fever. In one embodiment, patients who develop vasospasm have higher white blood cell counts, or in another embodiment, circulating immune complexes, or in another embodiment complement factors compared to patients who do not develop vasospasm. In one embodiment, cell adhesion molecules necessary for leukocyte-endothelial cell binding, including ICAM-1, are upregulated following SAH. In another embodiment, ICAM-1 levels are elevated in both the serum and the cerebrospinal fluid in patients who develop vasospasm, and predict a poor outcome following SAH. In one embodiment, this upregulation is associated with the extravasation of macrophages/neutrophils into the adventitia of blood-exposed vessels and the use of monoclonal antibodies against these cell adhesion molecules decreases in another embodiment, macrophage/neutrophil infiltration and prevents vasospasm. In another embodiment, Endothelin (ET) is a family of 3 vasoconstrictor isopeptides with common structural features (ET-1, ET-2, ET-3) that is expressed by macrophages, contributing to vasospasm severity.

Hp is a serum protein that in one embodiment, determines the extent of inflammation following a hemorrhagic event, such as SAH in one embodiment. After a hemorrhage, blood is extravasatedo into the interstitial space, leading in one embodiment, to the breakdown of erythrocytes and subsequent release of hemoglobin (Hb). Hp binds in another embodiment to free, extracorpuscular Hb and in one embodiment, promotes its clearance via the CD163 scavenger receptor that is present on macrophages. In one embodiment, binding and clearance of Hb neutralizes its oxidative and inflammatory potential.

In one embodiment, extracorpuscular Rb is a pro-inflammatory stimulus that upregulates the expression of endothelial and leukocyte adhesion molecules, thereby recruiting macrophages and neutrophils to the site of hemorrhage. Free Hb contributes in another embodiment, indirectly to inflammation by catalyzing the oxidation of arachidonic acid and promoting prostaglandin synthesis. In one embodiment, free Hb binds to nitric oxide (NO) and prevents NO-induced vasodilation. In another embodiment, the heme iron component of Hb promotes the accumulation of cell-damaging oxygen radicals and lipid peroxides by means of the Fenton reaction. In one embodiment, Hp plays a pivotal role in neutralizing Hb-induced inflammation and subsequent vasospasm associated with SAH in one embodiment, and other hemorrhagic events in other embodiments.

Accordingly and in one embodiment, provided herein is a method of providing a prognosis for development of vasospasm in a subject, comprising the steps of: obtaining a biological sample from a subject following a hemorrhagic event; determining the Haptoglobin (Hp) genotype in the biological sample, whereby a subject expressing a Hp-2-2 genotype has a high risk of developing vasospasm. In one embodiment, the term “prognosis” in any grammatical form refers to prediction of a pathological outcome, e.g., whether the subject suffering from the underlyiong pathology is likely to improve or regress.

Haptoglobin is inherited by two co-dominant autosomal alleles situated on chromosome 16 in humans, these are Hp1 and Hp2. There are three phenotypes Hp1-1, Hp2-1 and Hp2-2. Haptoglobin molecule is a tetramer comprising of four polypeptide chains, two alpha and two beta chains, of which the alpha chain is responsible for polymorphism since it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp 1-1 individuals have greater hemoglobin binding capacity when compared to those individuals with Hp2-1 and Hp2-2.

Hp in subjects with the Hp 1-1 phenotype is able to bind more hemoglobin on a Molar basis than Hps containing products of the haptoglobin 2 allele. Haptoglobin molecules in subjects with the haptoglobin 1-1 phenotype are also more efficient antioxidants, since the smaller size of haptoglobin 1-1 facilitates in one embodiment, its entry to extravascular sites of oxidative tissue injury compared to products of the haptoglobin 2 allele. In another embodiment, this also includes a significantly greater glomerular sieving of haptoglobin in subjects with Hp-1-1 phenotype.

In one embodiment, the Hp genotype determines the susceptibility to vasospasm, since the Hp 2-2 protein is inferior to Hp 1-1 in its Hb-clearing capacity, as well as its anti-inflammatory, immunomodulatory, and vasodilatory effects in other embodiments. In one embodiment, after a hemorrhagic event, the inflammation induced by extracorpuscular Rb is more intense in Hp 2-2 individuals. This increased inflammation results in another embodiment, in a more severe vasospasm and ischemia, causing clinically-relevant constitutional and neurologic symptoms.

In one embodiment, Hp 2-2 expressing individuals develop severe angiographic and clinical vasospasm, Hp 2-1 expressing individuals develop angiographic vasospasm without symptoms, and Hp 1-1 expressing individuals have no angiographic or clinical vasospasm following SAH. Interestingly, the distribution of patients who suffer symptomatic vasospasm (30%), angiographic vasospasm without symptoms (50%), and no angiographic or symptomatic vasospasm (20%) approximates the distribution of these Hp genotypes in the western world.

In western populations, 36% of individuals are Hp 2-2, 48% are Hp 2-1, and 16% are Hp 1-1. In one embodiment, the presence of the Hp 2 allele is associated with a higher rate of ultrasound-detected vasospasm.

The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined in one embodiment, from 10 μl of plasma by gel electrophoresis.

In one embodiment, the term “severe vasospasm” refers to significantly reduced lumen patencies. In another embodiment, severe vasospasm refers to decreased activity levels. In another embodiment, severe vasospasm refers to increased macrophage/neutrophil counts. In another embodiment, severe vasospasm refers to a combination of all symptoms described herein.

In one embodiment, the methods and systems of providing a prognosis for development of vasospasm in a subject, comprising the steps of: obtaining a biological sample from a subject following a hemorrhagic event; determining the Haptoglobin (Hp) genotype in the biological sample, allow for the selective early administration of potentially toxic treatments to only those patients with the Hp 2-2 genotype, who would clearly benefit from early aggressive treatment.

In another embodiment, the methods and systems of providing a prognosis for development of vasospasm in a subject provided herein have implications for stroke, where inflammation is a critical component of pathogenesis, and other conditions associated with inflammation-induced vasospasm, including traumatic brain injury in one embodiment, or craniotomy for tumors, meningitis, or their combination in other embodiments of the invention provided herein. In one embodiment, the methods and systems provided herein lead to the development of new therapeutic modalities to reduce the morbidity and mortality associated with vasospasm.

In one embodiment, early and in another embodiment, late vasospasm, involving the large basal intracranial arteries (middle cerebral and basilar), is a significant entity in head trauma, occurring in up to 25% of patients with head injury. Onset of vasospasm occurs in certain embodiments; from 48 hours to 7 days after a traumatic head injury. In another embodiment, ischemia associated with vasospasm impairs the metabolic need of the brain, initiating in another embodiment, multiple mechanisms of toxic metabolite formation and cell destruction. In one embodiment, provided herein is a method of providing a prognosis for development of vasospasm in a subject, comprising the steps of: obtaining a biological sample from a subject following a traumatic head injury; determining the Haptoglobin (Hp) genotype in the biological sample, whereby a subject expressing a Hp-2-2 genotype has a high risk of developing vasospasm as a result of the traumatic head injury.

Reperfusion following resolution vasospasm resulting from traumatic head injury, leads in one embodiment to additional neurological injury such as phagocytic damage to the endothelium and surrounding tissues in one embodiment, or release of oxygen-derived free radicals (ROS) and their combination in other embodiments. In one embodiment, ROS creates damage to vascular, neuronal, and glial membranes, with excitotoxic, intracellular calcium overload and excitatory amino acid release, glutamate, overwhelming in another embodiment, the antioxidant enzyme mechanisms of the affected cells, such as glutathione peroxidase in one embodiment.

In one embodiment, cerebral vasospasm occurs after cranial base tumor resection. In another embodiment, vasospasm manifest clinically; 1 to 30 days postoperatively, with most patients being symptomatic within 7 days. Symptoms include in one embodiment altered mental status, hemiparesis, monoparesis or their combination. In one embodiment, tumor size, or total operative time, vessel encasement, vessel narrowing, preoperative embolization, or their combination are factors that correlate with a higher incidence of vasospasm in addition to Hp genotype. Accordingly, provided herein is a method of providing a prognosis for development of vasospasm in a subject, comprising the steps of: obtaining a biological sample from a subject following cranial base tumor resection; determining the Haptoglobin (Hp) genotype in the biological sample, whereby a subject expressing a Hp-2-2 genotype has a high risk of developing vasospasm; and providing the prognosis based on the subject's haptoglobin genotype. In one embodiment, patients undergoing cranial base tumor resection, prediagnosed as expressing the Hp-2-2 allele, are treated aggressively with hypertensive, hypervolemic, hemodilutional therapy and early angioplasty. In another embodiment, in addition to Hp genotyping, the method of providing a prognosis for development of vasospasm in a subject, further comprise determining the Haptoglobin (Hp) genotype in a biological sample obtained from a subject following cranial base tumor resection, as well as determining tumor size, or total operative time, vessel encasement, vessel narrowing, preoperative embolization, or their combination whereby a subject expressing a Hp-2-2 genotype, a large tumor size, longer operative time, a more substantial vessel narrowing, lower vessel encasing or a combination thereof, has a higher risk of developing vasospasm; and providing the prognosis based on the subject's haptoglobin genotype.

Meningitis refers in one embodiment, to the inflammation of the meninges that results in the occurrence of meningeal symptoms such as headache in one embodiment, or nuchal rigidity, photophobia, an increased number of white blood cells in the cerebrospinal fluid (CSF), i.e., pleocytosis in other embodiments. Depending on the duration of symptoms, meningitis may be classified as acute in one embodiment or chronic in another. Acute meningitis denotes the evolution of symptoms within hours to several days, while chronic meningitis has an onset and duration of weeks to months. The duration of symptoms of chronic meningitis characteristically is no less than 4 weeks. In many instances, these syndromes overlap because they share many etiologic agents. In one embodiment, exposure of cells such as the endothelium in one embodiment, or meningeal macrophages and their combination in other embodiments, to bacterial products released during replication and death incites the synthesis of cytokines and proinflammatory mediators. The bacteria settles in certain embodiment in the subarachnoidal space, resulting in one embodiment, in vasculitis, which leads to narrowing and/or thrombosis of cerebral blood vessels and the propensity for ischemia and/or infarction of underlying brain. In one embodiment, meningitis results in the development of increased intracranial pressure (ICP), which may in certain embodiments lead to hemorrhagic events resulting in vasospasm. The pathophysiology of ICP involves many proinflammatory molecules as well as mechanical elements.

Vasospasm occurs in another embodiment, secondary to release of humoral factors elaborated within the CSF or blood vessel wall and in another embodiment would subsequently lead to vasodilatation or organic stenosis or both later in the course of disease. In one embodiment, Hp genotype is an independent risk factor in the development of vasosapsm resulting from bacterial or viral meningitis and the systems and methods described herein, are used in the providing of diagnosis and selection of optimal course of treatment.

In one embodiment, vasospasm occurs as the result of a brain aneurism, resulting from the rupture of plaque in a blood vessel in the subarachnoid space. In another embodiment, the methods provided herein are effective in the diagnosis and prognosis of development of vasospasm following an aneurism and their subsequent treatment.

According to one embodiment of the methods provided herein, determining the haptoglobin phenotype of a subject is effected by any one of a variety of methods including, but not limited to, a signal amplification method, a direct detection method and detection of at least one sequence change. These methods determine a phenotype indirectly, by determining a genotype. As will be explained hereinbelow, determination of a haptoglobin phenotype may also be accomplished directly by analysis of haptoglobin gene products.

Accordingly and in one embodiment, provided herein are methods and systems for providing a prognosis for development of vasospasm in a subject, comprising the steps of: obtaining a biological sample from a subject following a hemorrhagic event; determining the Haptoglobin (Hp) genotype in the biological sample, whereby said step of determining said haptoglobin genotype is effected by a signal amplification method, a direct detection method, a detection of at least one sequence change, an immunological method or a combination thereof.

In another embodiment, the methods and systems provided herein for providing a prognosis for development of vasospasm in a subject, comprising the steps of: obtaining a biological sample from a subject following a hemorrhagic event; determining the Haptoglobin (Hp) genotype in the biological sample is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the signal amplification methods provided herein, which in another embodiment, can be carried out using the systems provided herein, may amplify a DNA molecule or an RNA molecule. In another embodiment, signal amplification methods used as part of the present invention include, but are not limited to PCR, LCR (LAR), Self-Sustained Synthetic Reaction (3SR/NASBA) or a Q-Beta (Q.beta.) Replicase reaction.

Polymerase Chain Reaction (PCR): The polymerase chain reaction (PCR), refers in one embodiment to a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, in one embodiment, they are said to be “PCR-amplified.”

Ligase Chain Reaction (LCR or LAR): The ligase chain reaction [LCR; referred to, in another embodiment as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed in one embodiment and DNA ligase is added to the mixture. Provided that there is complete complementarity at the junction, ligase will covalently link each set of hybridized molecules. In another embodiment of LCR, two probes are ligated together only when they base-pair with sequences in the target sample, without gaps or mismatches. Repeated cycles of denaturation, and ligation amplify a short segment of DNA. LCR has is used in combination with PCR in one embodiment, to achieve enhanced detection of single-base changes. In another embodiment, because the four oligonucleotides used in this assay can pair to form two short ligatable fragments, there is the potential for the generation of target-independent background signal. The use of LCR for mutant screening is limited in another embodiment, to the examination of specific nucleic acid positions.

Self-Sustained Synthetic Reaction (3SR1NASBA): The self-sustained sequence replication reaction (3SR) refers in one embodiment, to a transcription-based in vitro amplification system that can exponentially amplify RNA sequences at a uniform temperature. The amplified RNA is utilized in certain embodiments, for mutation detection. In an embodiment of this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In one embodiment of the method, a probe which recognizes the sequence of interest is attached to the replicatable RNA template for Qβ. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

In another embodiment, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

In another embodiment, the methods and systems provided herein for providing a prognosis for development of vasospasm in a subject, comprising the steps of: obtaining a biological sample from a subject following a hemorrhagic event; determining the Haptoglobin (Hp) genotype in the biological sample that is effected by a direct detection method such as a cycling probe reaction (CPR), or a branched DNA analysis, or a combination thereof in other embodiments.

The direct detection method according to one embodiment is a cycling probe reaction (CPR) or a branched DNA analysis. When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

In another embodiment, the methods and systems provided herein for providing a prognosis for development of vasospasm in a subject, comprising the steps of: obtaining a biological sample from a subject following a hemorrhagic event; determining the Haptoglobin (Hp) genotype in the biological sample is effected by at least one sequence change, which employs in one embodiment a restriction fragment length polymorphism (RFLP analysis), or an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF) or their combination in other embodiments.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Determination of a haptoglobin phenotype may, as if further exemplified in the Examples section that hereinbelow, may be accomplished directly in one embodiment, by analyzing the protein gene products of the haptoglobin gene, or portions thereof. Such a direct analysis is often accomplished using an immunological detection method. In one embodiment, the methods and systems provided herein for providing a prognosis for development of vasospasm in a subject, comprising the steps of: obtaining a biological sample from a subject following a hemorrhagic event; determining the Haptoglobin (Hp) genotype in the biological sample by an immunological detection method, such as is a radio-immunoassay (RIA) in one embodiment, or an enzyme linked immunosorbent assay (ELISA), a western blot, an immunohistochemical analysis, or fluorescence activated cell sorting (FACS), or a combination thereof in other embodiments.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with I.sup. 125) immobilized on a precipitable carrier such as agarose beads. The number of counts in the precipitated pellet is proportional to the amount of substrate. In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

In one embodiment, provided herein is a method of providing a prognosis for development of vasospasm in a subject, comprising the steps of: obtaining a biological sample from a subject following a hemorrhagic event; determining the Haptoglobin (Hp) genotype in the biological sample, whereby a subject expressing a Hp-2-2 genotype has a high risk of developing vasospasm; providing the prognosis based on the subject's haptoglobin genotype; and determining the importance of reducing oxidative stress in the subject.

In one embodiment, the methods described herein, are effected by the systems provided herein. Accordingly and in one embodiment, provided herein is a system for providing a prognosis for development of vasospasm in a subject, comprising: a reagent, a packaging material; and instructions for determining the subject's Haptoglobin genotype. In one embodiment, a subject affected by a hemorrhagic event, expressing Hp-2-2 allele is at a high risk of developing vasospasm and the detection of the Hp-2-2 allele is done using the reagents and instructions comprised in the systems provided herein.

In one embodiment, the hemorrhagic event, for which the prognosis of vasospasm development is sought, is a traumatic brain injury, a craniotomy for tumors, a meningitis, a subarachnoid hemorrhage (SAH), or their combination. In one embodiment, the systems provided herein further comprise reagents and instructions for determining other risk factors associated with the hemorrhagic events described herein.

In one embodiment the systems for providing a prognosis for development of vasospasm in a subject, comprising: a reagent, a packaging material; and instructions for determining the subject's Haptoglobin genotype, may further comprise standards, or in another embodiment, additional reagents and instructions for determining the importance of reducing oxidative stress in the subject.

In one embodiment, the systems provided herein are used to carry out any of the methods described herein for genotyping Haptoglobin used in the prognosis of developing vasospasm. The “prognosis” refers in another embodiment, to a forecast as to the probable outcome of vasospasm resulting from a hemorrhagic event; the prospect as to recovery from the event as indicated by the nature and symptoms of the case.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects. In one embodiment, the subject is diabetic.

Embodied herein are methods for treatment of vasospasm as a consequence of SAH, and compounds and compositions thereof useful therefor. In another embodiment a subject whose Hp genotype is 2-2 will benefit more from treatment as described herein. In one embodiment, because molecular oxygen is virtually everywhere and it freely accepts electrons, oxygen-centered radicals are the most common mediators of cellular free radical reactions. Accordingly and in one embodiment, provided herein are methods and compositions for treating, or in another embodiment, methods and compositions for inhibiting or suppressing, or in another embodiment, methods and compositions for reducing symptoms of vasospasm in a subject, where the subject suffered a hemorrhagic event, comprising the step of contacting the subject with the compositions of the invention, which comprise a therapeutically effective amount of an antioxidant. In another embodiment, provided herein are methods and compositions for treating, or in another embodiment, methods and compositions for inhibiting or suppressing, or in another embodiment, methods and compositions for reducing symptoms of vasospasm in a subject, where the subject suffered a hemorrhagic event, comprising the step of contacting the subject with the compositions of the invention, which comprise a therapeutically effective amount of glutathione peroxidase mimetic or its isomer, metabolite, and/or salt therefore. In another embodiment, the subject's Hp genotype is determined prior to treatment and treatment initiated or more aggressively initiated and provided in a subject with Hp 2-2.

As noted above, in one embodiment, early and late vasospasm, involving the large basal intracranial arteries (middle cerebral and basilar), is a significant entity in head trauma, occurring in up to 25% of patients with head injury. Onset of vasospasm occurs in certain embodiments; from 48 hours to 7 days after a traumatic head injury. In another embodiment, cerebral ischemia associated with vasospasm impairs the metabolic need of the brain, initiating in another embodiment, multiple mechanisms of toxic metabolite formation and cell destruction. Accordingly provided herein is a method of treating a vasospasm in a subject, comprising: contacting said subject, wherein the subject has suffered a traumatic head trauma with an effective amount of a composition comprising glutathione peroxidase mimetic or its isomer, metabolite, and/or salt therefore between about 36 to about 6 days post trauma, thereby treating vasospasm. In another embodiment, the subject is treated with an effective amount of an antioxidant.

In another embodiment, the term “Cerebral Ischemia” or “cerebral ischemic” or “a cerebral ischemic condition” refer to a medical event which is pathological in origin, or to a surgical intervention which is imposed on a subject, wherein circulation to a region of the brain is impeded or blocked, either temporarily, as in vasospasm or transient ischemic attack (TIA) or permanently, as in thrombolic occlusion. The affected region is deprived of oxygen and nutrients as a consequence of the ischemic event. This deprivation leads to the injuries of infarction or in the region affected. In another embodiment, ischemia occurs in the brain during a thromboembolic stroke, hemorrhagic stroke, cerebral vasospasm, head trauma, cardiac arrest, severe blood loss due to injury or internal hemorrhage and other similar conditions that disrupt normal blood flow. In another embodiment, it may also occur after a head trauma, since the pressure caused by edema presses against and flattens the arteries and veins inside the brain, thereby reducing their ability to carry blood through the brain. Cerebral ischemia may also occur as a result of macro- or micro-emboli, such as may occur subsequent to cardiopulmonary bypass surgery. In one embodiment, cerebral ischemia and hemorrhagic event are used interchangeably.

In another embodiment, reperfusion following resolution vasospasm resulting from traumatic head injury, leads in one embodiment to additional neurological injury such as phagocytic damage to the endothelium and surrounding tissues in one embodiment, or release of oxygen-derived free radicals (ROS) and their combination in other embodiments. In one embodiment, ROS creates damage to vascular, neuronal, and glial membranes, with excitotoxic, intracellular calcium overload and excitatory amino acid release, glutamate, overwhelming in another embodiment, the antioxidant enzyme mechanisms of the affected cells, such as glutathione peroxidase in one embodiment. In one embodiment, the therapeutically effective amount of a composition comprising glutathione peroxidase mimetic or its isomer, metabolite, and/or salt therefore, used in the methods described herein, is effective in removing the ROS from the interstitial subarachnoid space, thereby reducing damage from reperfusion following the resolution of vasospasm.

In one embodiment, cerebral vasospasm occurs after cranial base tumor resection. Vasospasm manifest clinically; 1 to 30 days postoperatively, with most patients being symptomatic within 7 days. Symptoms include in one embodiment altered mental status, hemiparesis, monoparesis or their combination. In one embodiment, Haptoglobin genotype, tumor size, or total operative time, vessel encasement, vessel narrowing, preoperative embolization, or their combination are factors that correlate with a higher incidence of vasospasm. In one embodiment, the methods and compositions provided herein are effective in treating vasospasm resulting from cranial base tumor resection. Hydrogen peroxide (HP) is routinely used during neurosurgical procedures to augment hemostasis after intracranial tissue resection, where it irreversibly damages mesothelial and neural tissue. Using the compositions and methods described herein, in one embodiment damage done through the use of HP during cranial tumors resection is ameliorated. Accordingly and in one embodiment, provided herein is a method of treating a vasospasm in a subject, comprising: contacting said subject via intracranial administration, wherein the subject is undergoing cranial tumor resection in one embodiment, or has undergone cranial tumor resection, with an effective amount of a composition comprising glutathione peroxidase or its isomer, metabolite, and/or salt therefore between about 36 to about 6 days post trauma, thereby treating vasospasm. In another embodiment, the glutathione peroxidase or its isomer, metabolite, and/or salt therefore is represented by the compounds of formula I-X.

Survivors of bacterial meningitis suffer in one embodiment, from a broad spectrum of neurologic sequelae that arise from neuronal cell damage. Pneumococcus is the most common and most aggressive human meningeal pathogen, causing death in up to 30% of cases and neurologic sequelae in 30-50% of survivors. Permanent loss of neurons by the induction of apoptosis in the hippocampus contributes in another embodiment to the outcome.

Meningitis refers in one embodiment, to the inflammation of the meninges that results in the occurrence of meningeal symptoms such as headache in one embodiment, or nuchal rigidity, photophobia, an increased number of white blood cells in the cerebrospinal fluid (CSF), i.e., pleocytosis in other embodiments. Depending on the duration of symptoms, meningitis may be classified in one embodiment as acute or chronic. Acute meningitis denotes the evolution of symptoms within hours to several days, while chronic meningitis has an onset and duration of weeks to months. The duration of symptoms of chronic meningitis characteristically is no less than 4 weeks. In many instances, these syndromes overlap because they share many etiologic agents. In one embodiment, exposure of cells such as the endothelium in one embodiment, or meningeal macrophages and their combination in other embodiments, to bacterial products released during replication and death incites the synthesis of cytokines and proinflammatory mediators. The bacteria settles in certain embodiment in the subarachnoidal space, resulting, in vasculitis, which leads to narrowing and/or thrombosis of cerebral blood vessels and the propensity for ischemia and/or infarction of underlying brain. In one embodiment, meningitis results in the development of increased intracranial pressure (ICP), which may in certain embodiments lead to hemorrhagic events resulting in vasospasm. The pathophysiology of ICP involves many proinflammatory molecules as well as mechanical elements.

In one embodiment, host-derived NO and bacterium-derived HP contribute to the death of hippocampal neurons in bacterial meningitis (BM), and become most prominent in the interplay between pneumococcal oxidative and eukaryotic nitrogen intermediates, leading to the formation of peroxynitrite. HP rapidly diffuses through eukaryotic cell membranes to damage intracellular targets (e.g., mitochondria and DNA) and to trigger apoptosis. In one embodiment, pneumococcal-derived HP is a bacterial factor contributing to increases of intracellular ROS and Ca²⁺ and release of AIF. In one embodiment, the oxidative damage, or oxidative stress associated with BM is inhibited by treatment with antioxidants reducing cerebral ischemic damage and preventing cerebral blood flow reduction. In another embodiment, glutathione peroxidase is the only oxygen scavenger enzyme that is continuously upregulated in the early and late phases of acute BM, indicating the need for its activity.

In one embodiment, the production of superoxide anions (O2.-), hydrogen peroxide (HP) and malondialdehyde (MDA) and the activities of xanthine oxidase (XO), superoxide dismutase (SOD) and glutathione peroxidase (GPx) were found to be significantly increased in children with acute bacterial meningitis (ABM) or tuberculous meningitis (TBM) who died, indicating that natural or synthetic antioxidants may prevent disease progression and tissue damage in childhood meningitis. Accordingly, in one embodiment, provided herein is a method of preventing disease progression and tissue damage in childhood meningitis, comprising the step of contacting a child suffering from acute bacterial meningitis (ABM) or tuberculous meningitis (TBM), via parenteral administration, with a composition comprising glutathione peroxidase or its isomer, metabolite, and/or salt therefore.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues such as glial cells and neurons, must be able to protect themselves against the toxic effects of ROS. In one embodiment ROS-detoxifying enzymes have an important role in epithelial wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O⁻₂⁻); nitric oxide (NO⁻) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of O⁻₂⁻ and H₂O₂. First, SOD catalyses the dismutation of O⁻₂⁻ to H₂O₂ and molecular oxygen (O₂), resulting in selective O⁻₂⁻ scavenging. Then, GPX and catalase independently decompose H₂O₂ to H₂O. In another embodiment, ROS is released from the active neutrophils in the inflammatory tissue, attacking DNA and/or membrane lipids and causing chemical damage, including in one embodiment, to healthy tissue. When free radicals are generated in excess or when FRSEs are defective, H₂O₂ is reduced into hydroxyl radical (OH⁻), which is one of the highly reactive ROS responsible in one embodiment for initiation of lipid peroxidation of cellular membranes. In another embodiment, organic peroxide-induced lipid peroxidation is implicated as one of the essential mechanisms of toxicity in the death of hippocampal neurons. In one embodiment, an indicator of the oxidative stress in the cell is the level of lipid peroxidation and its final product is MDA. In another embodiment the level of lipid peroxidation increases in inflammatory diseases, such as meningitis in one embodiment. In one embodiment, the compounds provided herein and in another embodiment, are represented by the compounds of formula I-X, are effective antioxidants, capable of reducing lipid peroxidation, or in another embodiment, are effective as anti-inflammatory agents.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

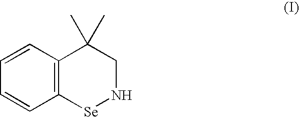

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of π electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet. In vitro and in vivo studies with the compound of formula 1, a show in one embodiment, that glutahion peroxidase or its isomer, metabolite, and/or salt therefore is capable of protecting cells against reactive oxygen species.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx or its pharmaceutically acceptable salt, its functional derivative, its synthetic analog or a combination thereof, is used in the methods and compositions of the invention.

In one embodiment, the glutathione peroxidase, is represented by formula I:

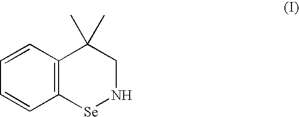

In one embodiment, the compound of formula (II), refers to benzisoselen-azoline or -azine derivatives represented by the following general formula:

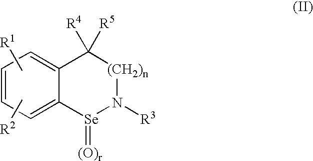

where: R¹, R²=hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂NR⁶R⁷; —CO(CH₂)ₚCOR⁸; R⁹; R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸; R⁴=lower alkyl; aralkyl; substituted aralkyl; —(CH₂)ₚCOR⁸; —(CH₂)ₚR⁸; F; R⁵=lower alkyl; aralkyl; substituted aralkyl; R⁶=lower alkyl; aralkyl; substituted aralkyl; —(CH₂)ₘCOR⁸; —(CH₂)_{q}R⁸; R⁷=lower alkyl; aralkyl; substituted aralkyl; —(CH₂)ₘCOR⁸; R⁸=lower alkyl; aralkyl; substituted aralkyl; aryl; substituted aryl; heteroaryl; substituted heteroaryl; hydroxy; lower alkoxy; R⁹; R⁹=

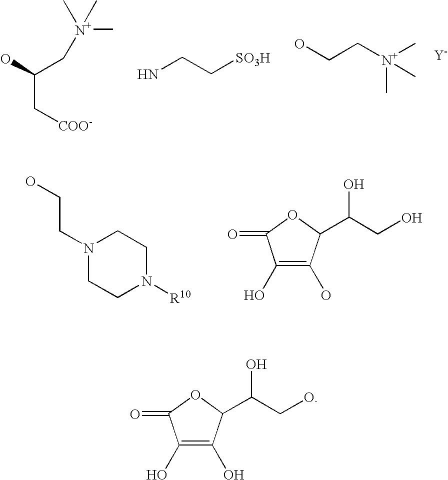

R¹⁰=hydrogen; lower alkyl; aralkyl or substituted aralkyl; aryl or substituted aryl. Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl). or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —ORₓ; —SRₓ; —S(O)Rₓ—S(O)₂Rₓ; —NRₓ, —(CO)Rₓ; —N(Rₓ)CO₂Rₓ; —N(Rₓ)S(O)₂Rₓ; —N(Rₓ)C(O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety.

In one embodiment, the glutathione peroxidase or its isomer, metabolite, and/or salt therefore, used in the methods and composition provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating vasospasm associated with pathologies resulting from hemorrhagic event, comprising organoselenium compounds, thereby increasing endogenous anti-oxidant ability of the cells, or in another embodiment, scavenging free radicals causing apoptosis of hippocampal neurons and their associated pathologies.

Accordingly and in another embodiment, provided herein is a composition for treating a vasospasm in a subject wherein the subject suffered a hemorrhagic event, comprising: a therapeutically effective amount of a composition comprising glutathione peroxidase or its isomer, metabolite, and/or salt therefore.

In another embodiment, the glutathione peroxidase or its isomer, metabolite, and/or salt therefore used in the compositions and methods provided herein, is represented by the compound of formula III:

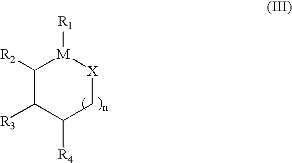

wherein,
the compound of formula I is a ring; and
   X is O or NH
   M is Se or Te
   n is 0-2
   R₁ is oxygen; and
forms an oxo complex with M; or
   R₁ is oxygen or NH; and
forms together with the metal, a 4-7 member ring, which optionally is substituted by an oxo group; or
forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
R₂, R₃ and R₄ are independently hydrogen, alkyl, oxo, amino or together with the organometallic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
   if R₄ is an oxo group, and R₂ and R₃ form together with the organometalic ring a fused benzene ring, R₁ is oxygen, n is 0 and forms together with the metal a first 5 member ring, substituted by an oxo group a to R₁, and said ring is fused to a second benzene ring, then M is Te.

In one embodiment, a 4-7-member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7-member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In another embodiment, a compound of formula IV is provided, wherein M, R₁ and R₄ are as described above for formula III.

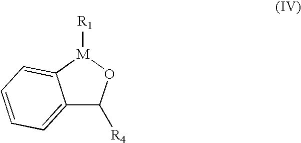

In another embodiment, a compound of formula V is provided, wherein M, R₂, R₃ and R₄ are as described above for formula III.

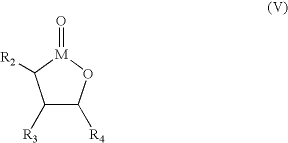

In another embodiment, a compound of formula VI is provided, wherein M, R₂, R₃ and R₄ are as described above for formula III.

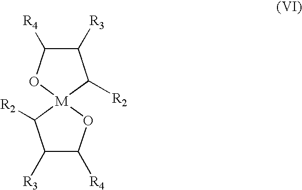

In another embodiment, a compound of formula (VII) is provided, wherein M, R₂ and R₃ are as described above for formula III

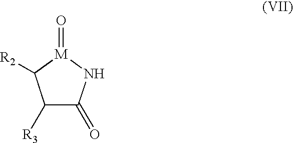

In another embodiment, a compound of formula VIII is provided, wherein M, R₂ and R₃ are as described above for formula III.

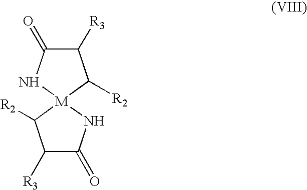

In one embodiment, the compound of formula III, used in the compositions and methods provided herein, is represented by any one of the following compounds or their combinations:

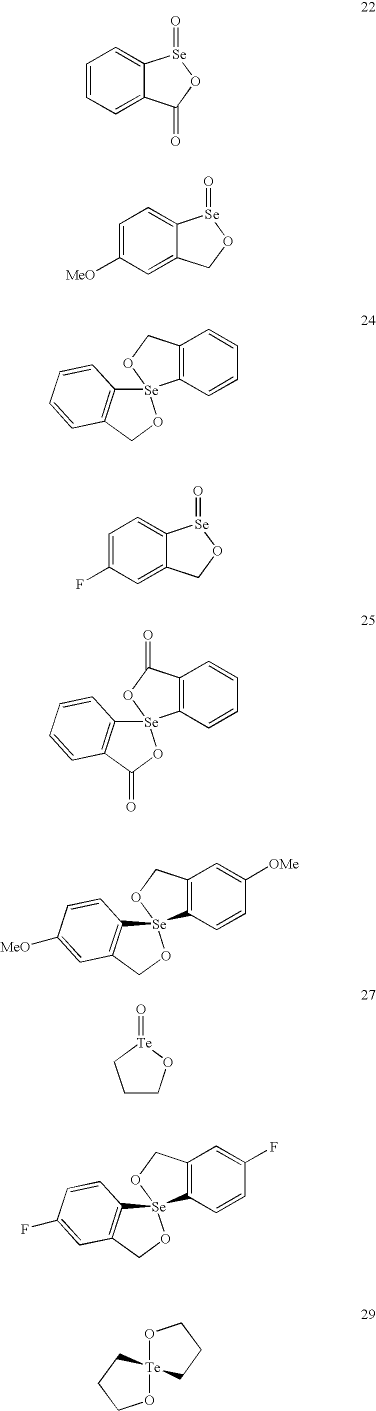

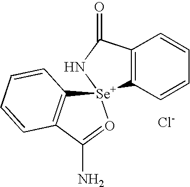

In another embodiment, the glutathione peroxidase or its isomer, metabolite, and/or salt therefore used in the compositions and methods provided herein, is represented by the compound of formula IX:

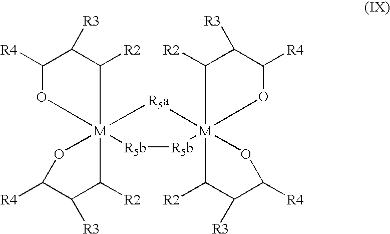

wherein,

M is Se or Te;

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the therapeutic value of the primary agents described above in the compositions provided herein, can be further augmented by administration in conjunction with recognized antioxidant free radical trapping compounds such as α-tocopherol, edaravone or other co-agents previously recognized as adjuncts which facilitate in vivo capability to inhibit lipid peroxidation.

In one embodiment agents which function to supplement the chain-breaking antioxidant property of vitamin E are ubiquinol, or seleno-amino acids and sulfhydryl compounds (e.g., glutathione, sulfhydryl proteins, cysteine and methionine) in other embodiments. Other substances in this general group include in other embodiments: butylated hydroxytoluene (BHT), butylated hydroxyanisole (BHA), propyl gallate (PG), dodecylgallate, tert-butylhydroquinone (TBHQ), dihydrolipoic acid, prostaglandin B₁ oligomers (also known as polymeric 15-keto prostaglandin B or PGBₓ), 2-aminomethyl-4-tert-butyl-6-iodophenol, 2-aminomethyl-4-tert-butyl-6-propionylphenol, 2,6-di-tert-butyl-4-[2′-thenoyl]phenol, N,N′-diphenyl-p-phenylenediamine, ethoxyquin, probucol and its derivative such as AGI-1067, 5-[[3,5-bis(1,1-dimethylethyl)-4-hydroxyphenyl]methylene]-3-(dimethylamino)-4-thiazolidinone (LY221068), 5-[[3,5-bis(1,1-dimethylethyl)-4-hydroxyphenyl]meth-ylene]-3-(methylamino)-4-thiazolidinone (LY269415), D-myoinositol-1,2,6-trisphosphate, nordihydroguaiaretic acid, deferoxamine mesylate, tirilazad mesylate (U-74006F), derivative of tirilazad in which the steroid portion of the chemical structure has been replaced with the tetramethyl chroman portion of d-α-tocopherol (U78517F), trimetazidine, N,N′-dimethylthiourea, 2-(2-hydroxy-4-methylphenyl)aminothiazolehydrochloride, or 2-L-oxothiazolidine.

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

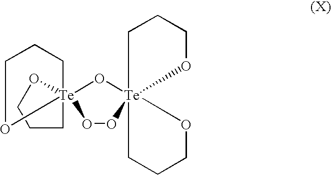

In one embodiment, the compounds represented by formula I-X, mimic the in-vivo activity of glutathione peroxidase. The term “mimic” refers, in one embodiment to comparable, identical, or superior activity, in the context of conversion, timing, stability or overall performance of the compound, or any combination thereof.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

In another embodiment, Thioctic acid, also known as α-lipoic acid, is used as an antioxidant in the compositions and methods provided herein, including its sodium salt and ethylenediamine derivatives. In one embodiment, antioxidants and free radical trapping substances used in the compositions and methods provided herein, are plant (e.g., vegetable) active ingredients. This category, includes in one embodiment parthenolide, or lycopene, genistein, quercetin, morin, curcumin, apigenin, sesamol, chlorogenic acid, fisetin, ellagic acid, quillaia saponin, capsaicin, ginsenoside, silymarin, kaempferol, ginkgetin, bilobetin, isoginkgetin, isorhamnetin, herbimycin, rutin, bromelain, levendustin A, orerbstatin in other embodiments.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the compositions provided herein are used for the treatment of vasospasm conditions and may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In some embodiments, the compositions and methods provided herein permit direct application to the site where it is needed. In the practice of the methods provided herein, it is contemplated that virtually any of the compositions provided herein can be employed.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein are suitable for oral, intraoral, rectal, parenteral, topical epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with one or more therapeutic agents. These agents are in other embodiments, age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

In one embodiment, the compositions described herein, are used in the methods described herein. Accordingly and in another embodiment, provided herein is a method of treating a vasospasm in a subject, comprising: contacting said subject, wherein the subject has suffered a hemorrhagic event with an effective amount of a composition comprising glutathione peroxidase or its isomer, metabolite, and/or salt therefore, thereby treating vasospasm.

In one embodiment, the term “administering” refers to bringing a subject in contact with the compositions provided herein. For example, in one embodiment, the compositions provided herein are suitable for oral administration, whereby bringing the subject in contact with the composition comprises ingesting the compositions. A person skilled in the art would readily recognize that the methods of bringing the subject in contact with the compositions provided herein, will depend on many variables such as, without any intention to limit the modes of administration; the hemorrhagic event treated, age, pre-existing conditions, other agents administered to the subject, the severity of symptoms, location of the affected are and the like. In one embodiment, provided herein are embodiments of methods for administering the compounds of the present invention to a subject, through any appropriate route, as will be appreciated by one skilled in the art.

In one embodiment, the methods provided herein, using the compositions provided herein, further comprise contacting the subject with one or more additional therapeutic agent. In one embodiment, the additional agent is an antibiotic, or Ca++ channel blocker, or a metal chelator, or their combination in another embodiment.

In one embodiment, the additional agent is Probucol or its metabolites and derivatives. In one embodiment oxidative modification of LDL within the arterial wall is implicated in the early stages of atherogenesis, referring in another embodiment to the pathologic process that leads to occlusive arterial lesions principally responsible for myocardial and cerebral infarction, lesions principally responsible for myocardial and cerebral infarction, gangrene of the extremities, and subsequent loss of function. Oxidized LDL assists in another embodiment, in foam cell formation, and is cytotoxic, as well as induces various proatherogenic processes in other embodiments. Lipid peroxidation is one of the earliest processes occurring during LDL oxidation. In one embodiment, increasing antioxidant defense against such damage using the compositions described herein attenuates the initial stages of atherogenesis. In one embodiment, probucol or its isomer, metabolite, derivatives or their combination is effective in inhibiting the release of IL-1, increasing the expression of cholesterol ester transfer protein, or in modifying oxidative metabolism at the cell level. Accordingly, provided herein is a method of treating a vasospasm in a subject, comprising: contacting said subject, wherein the subject has suffered a hemorrhagic event with an effective amount of a composition comprising glutathione peroxidase or its isomer, metabolite, and/or salt therefore, and with one or more additional therapeutic agent wherein the additional agent is Probucol, or its isomer, metabolite, and/or salt therefore thereby treating vasospasm.

In one embodiment, foam cells are formed by the uncontrolled uptake of oxidized LDL (oxLDL) containing cholesterol and lipids. In another embodiment, the gene expression patterns are altered in atherosclerosis. In one embodiment, the genes responsible for such alteration are the scavenger receptors, which take up modified LDL, leading to foam cell formation at the atherogenic lesion, such as the glycoprotein CD36 in certain embodiment. The CD36 scavenger receptor is expressed in megakaryocytes/platelets, monocytes/macrophages, mammary epithelial cells, and adipocytes. It is also expressed in capillary endothelial cells of adipose, cardiac, and muscle tissue and at low levels in the vascular endothelium of the brain, lung, and kidneys. CD36 binds in one embodiment, to oxLDL. In another embodiment, CD36 is upregulated by the increases in blood concentration of oxLDL.

In another embodiment, the additional agent is an inhibitor of oxidized CD36. In one embodiment, class A type I and II macrophage scavenger receptors (SRA) and CD36 are the major receptors involved in foam cell formation, mediating in another embodiment, the influx of lipids into the macrophages and regulating fundamental macrophage functions, thereby playing a key role in atherosclerosis. Accordingly, provided herein is a method of treating a vasospasm in a subject, comprising: contacting said subject, wherein the subject has suffered a hemorrhagic event with an effective amount of a composition comprising glutathione peroxidase or its isomer, metabolite, and/or salt therefore, and with one or more additional therapeutic agent wherein the additional agent is an inhibitor of oxidized CD36, or its isomer, metabolite, and/or salt therefore thereby treating vasospasm.

In one embodiment, the additional agent is antibodies that bind up oxidized lipid and therefore reduce oxidized lipid levels. In one embodiment, circulating autoantibodies against oxidised LDL exist in human subjects and antibodies against oxidized LDL were found to be present in patients with cardiovascular disease. in another embodiment, immune reactions that protect against the development of atherosclerosis exists, which involve autoimmunity against oxidised LDL.

In another embodiment, peptides derived from apolipoprotein B-100 (ApoB-100) protect subjects from development of atherosclerosis. In another embodiment, antibodies that bind to oxidised epitopes present in LDL particles, protect from development of atherosclerotic plaques. Oxidised LDL contains several different epitopes that can be recognised by antibodies. LDL may undergo oxidative and degrading changes through a wide variety of different chemical reactions. These include reactions caused by different types of modifications caused by the activity of oxygen, enzymes (e.g. myeloperoxidase), metal ions (e.g. Fe²⁺ and Cu²⁺), free radicals and other types of chemical stress. In one embodiment, the additional agent used in the methods and compositions provided herein, is an anti-OxLDL antibody, such as those reported in WO/2004/030607, especially IEI-A8, IE1-D8, IE1-E3, IE1-G8, KTT-B8 and KTT-D6, are incorporated herein by reference, as well as BI-204.

Accordingly, provided herein is a method of treating a vasospasm in a subject, comprising: contacting said subject, wherein the subject has suffered a hemorrhagic event with an effective amount of a composition comprising glutathione peroxidase or its isomer, metabolite, and/or salt therefore, and with one or more additional therapeutic agent wherein the additional agent is an anti-OxLDL antibody, or its isomer, metabolite, and/or salt therefore thereby treating vasospasm.

In one embodiment, the compositions described herein, which, in another embodiment, are used in the methods provided herein, further comprise another therapeutic agent that is not an antioxidant or its isomer, metabolite, and/or salt therefore. In another embodiment, the agent that is not an antioxidant or its isomer, metabolite, and/or salt therefore, is an aldosterone inhibitor, and angiotensin-converting anzyme, an angiotensin receptor AT₁ blocker (ARB), an angiotensin II receptor antagonist, a calcium channel blocker, a diuretic, digitalis, a beta blocker, a statin, a cholestyramine, a NSAID, a glycation inhibitor or a combination thereof.

In one embodiment, the additional therapeutic agent used in the methods and compositions described herein is a statin. In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

Statins inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease.

Statins used in the compositions and methods of the invention are lovastatin (referred to as mevinolin in one embodiment, or monacolin-K in another embodiment), compactin (referred to as mevastatin in one embodiment, or ML-236B in another embodiment), pravastatin, atorvastatin (Lipitor) rosuvastatin (Crestor) fluvastatin (Lescol), simvastatin (Zocor), cerivastatin. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

In one embodiment, the additional agent may be an anti-dyslipidemic agent such as (i) bile acid sequestrants such as, cholestyramine, colesevelem, colestipol, dialkylaminoalkyl derivatives. of a cross-linked dextran; Colestid™; LoCholest™; and Questran™, and the like; (ii) HMG-CoA reductase inhibitors such as atorvastatin, itavastatin, fluvastatin, lovastatin, pravastatin, rivastatin, rosuvastatin, simvastatin, and ZD-4522, and the like; (iii) HMG-CoA synthase inhibitors; (iv) cholesterol absorption inhibitors such as stanol esters, beta-sitosterol, sterol glycosides such as tiqueside; and azetidinones such as ezetimibe, vytorin, and the like; (v) acyl coenzyme A-cholesterol acyl transferase (ACAT) inhibitors such as avasimibe, eflucimibe, KY505, SMP 797, and the like; (vi) CETP inhibitors such as JTT 705, torcetrapib, CP 532,632, BAY63-2149, SC 591, SC 795, and the like; (vii) squalene synthetase inhibitors; (viii) anti-oxidants such as probucol, and the like; (ix) PPAR.alpha. agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid™, Lopid™ and Tricor™, and the like; (x) FXR receptor modulators such as GW 4064, SR 103912, and the like; (xi) LXR receptor such as GW 3965, T9013137, and XTC0179628, and the like; (xii) lipoprotein synthesis inhibitors such as niacin; (xiii) renin angiotensin system inhibitors; (xiv) PPAR o partial agonists; (xv) bile acid reabsorption inhibitors, such as BARI 1453, SC435, PHA384640, S892.1, AZD7706, and the like; (xvi) PPAR.delta. agonists such as GW 501516, and GW 590735, and the like; (xvii) triglyceride synthesis inhibitors; (xviii) microsomal triglyceride transport (MTTP) inhibitors, such as inplitapide, LAB687, and CP346086, and the like; (xix) transcription modulators; (xx) squalene epoxidase inhibitors; (xxi) low density lipoprotein (LDL) receptor inducers; (xxii) platelet aggregation inhibitors; (xxiii) 5-LO or FLAP inhibitors; and (xiv) niacin receptor agonists.

In one embodiment, the additional agent administered as part of the compositions, used in the methods provided herein, is an anti-platelet agents (or platelet inhibitory agents). The term anti-platelet agents (or platelet inhibitory agents), refers in one embodiment to agents that inhibit platelet function by inhibiting the aggregation, or by adhesion or granular secretion of platelets in other embodiments. In another embodiment, the anti-platelet agents used in the compositions described herein include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. In another embodiment, the anti-platelet agent is IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof. In another embodiment, the term anti-platelet agents (or platelet inhibitory agents), refers to ADP (adenosine diphosphate) receptor antagonists, which is in one embodiment, an antagonists of the purinergic receptors P₂Y₁ and P₂Y₁₂. In one embodiment, P₂Y₁₂ receptor antagonists is ticlopidine, clopidogrel, or their combination and pharmaceutically acceptable salts or prodrugs thereof.

In another embodiment, the additional agent administered as part of the compositions, used in the methods provided herein, is an anti-hypertensive agents such as (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (ix) α/β adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XEN010, and the like; and (xi) -alpha 2 agonists such as lofexidine, tiamenidine, moxonidine, rilmenidine and guanobenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

The renin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme renin from the juxtaglomerular cells in the kidney activates in another embodiment, the renin-angiotensin-aldosterone system (RAAS), acting on a naturally-occurring substrate, angiotensinogen, to release in another embodiment, a decapeptide, Angiotensin I. Angiotensin converting enzyme (“ACE”) cleaves in one embodiment, the secreated decapeptide, producing an octapeptide, Angiotensin II, which is in another embodiment, the primary active species of the RAAS system. Angiotensin II stimulates in one embodiment, aldosterone secretion, promoting sodium and fluid retention, inhibiting renin secretion, increasing sympathetic nervous system activity, stimulating vasopressin secretion, causing a positive cardiac inotropic effect or modulating other hormonal systems in other embodiments.

A representative group of ACE inhibitors consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, 15B2, indolapril, ketomethylureas, KRI-1177, KRI-1230, L-681176, libenzapril, MCD, MDL-27088, MDL-27467A, moveltipril, MS-41, nicotianamine, pentopril, phenacein, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS182657, Asahi Chemical C-111, Asahi Chemical C-112, Dainippon DU-1777, mixanpril, Prentyl, zofenoprilat, 1-(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses Aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the Aldosterone, even when there are spare (unbound) Aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplerenone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a glutathione peroxidase or its isomer, functional derivative, synthetic analog, pharmaceutically acceptable salt or combination thereof, wherein the aldosterone antagonist is epoxymexrenone, or eplerenone, dihydrospirorenone, 2,2;6,6-diethlylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In another embodiment, the angiotensin II receptor antagonist used in the compositions and methods of the invention is losartan, irbesartan, eprosartan, candesartan, valsartan, telmisartan, zolasartin, tasosartan or a combination thereof. Examples of angiotensin II receptor antagonists used in the compositions and methods of the invention are in one embodiment biphenyltetrazole compounds or biphenylcarboxylic acid compounds or CS-866, losartan, candesartan, valsartan or irbesartan in other embodiments. In one embodiment, where the above-mentioned compounds have asymmetric carbons, the angiotensin II receptor antagonists of the compositions and methods used in the present invention are optical isomers and mixtures of said isomers. In one embodiment, hydrates of the above-mentioned compounds are also included.

In one embodiment, Cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca²⁺-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in cystolic pressure.

In one embodiment, calcium channel blockers, are amlodipine, aranidipine, barnidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, veraparmil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, angiotensin II receptor blocker (ARB) are used in the compositions and methods of the invention. Angiotensin II receptor blocker (ARB) refers in one embodiment to a pharmaceutical agent that selectively blocks the binding of AII to the AT₁ receptor. ARBs provide in another embodiment, a more complete blockade of the RAAS by preventing the binding of AII to its primary biological receptor (AII type 1 receptor [AT₁]).

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglycate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In another embodiment, the additional agent administered as part of the compositions, used in the methods provided herein, is a glycation inhibitor, such as pimagedine hydrochloride in one embodiment, or ALT-711, EXO-226, KGR-1380, aminoguanidine, ALT946, pyratoxanthine, N-phenacylthiazolium bromide (ALT766), pyrrolidinedithiocarbamate or their combination in yet another embodiment.

The following examples are presented in order to more fully illustrate the preferred embodiments of the invention. They should in no way be construed, however, as limiting the broad scope of the invention.

### EXAMPLES

### Materials and Methods

### Experimental Design

The purpose was to assess the role of the Hp genotype in the development of vasospasm following SAH in mice. This was accomplished by (1) inducing SAH, (2) determining the extent and manifestations of vasospasm, and (3) comparing the extent of vasospasm in Hp 1-1 mice to that of Hp 2-2 mice. The extent and manifestations of vasospasm were assessed by measuring the circumference of the basilar artery to determine lumen patency, quantifying the activity level, and counting the number of vessel-infiltrated macrophages/neutrophils.

Hp 1-1 and Hp 2-2 mice were separately randomized to three experimental groups each to assess the severity of their vasospasm following SAH. For the Hp 1-1 mice, one group underwent injection of autologous blood into the cisterna magna (n=15), a second group underwent injection of normal saline solution into the cisterna magna (n=15), and a third group underwent no procedures (n=15). The setup was the same for the Hp 2-2 mice, where one group underwent injection of autologous blood into the cisterna magna (n=15), a second group underwent injection of normal saline solution into the cisterna magna (n=15), and a third group underwent no procedures (n=15). In each group, 5 mice were used exclusively for macrophage/neutrophil immunohistochemical studies.

### Animals

C57B1/6J Hp 1-1 mice (Jackson Laboratories, Bar Harbor, Me.) and C57B1/6J Hp 2-2 mice (Technion Institute, Haifa, Israel) weighing between 22-27 grams were used.

The mice were housed in standard animal facilities with free access to Baltimore City water and rodent chow. The Johns Hopkins University School of Medicine Animal Care and Use Committee approved all experimental protocols.

### Construction of the Murine Hp 2 Allele

Wild type C57B1/6 mice contain only a class 1 Hp allele, which is over 90% homologous to the human Hp 1 allele, and therefore possess the Hp 1-1 genotype. Mice possessing the Hp 2-2 genotype had to be genetically engineered. A murine, genetically engineered Hp 2 allele was developed previously, by duplicating exons 3 and 4 of the Hp 1 allele, and targeted its insertion to the murine Hp locus by homologous recombination. This approach allowed for the study of the Hp 2 gene in its normal genomic location. The Hp serum concentration from Hp 2-2 and Hp 1-1 mice was similar, and the concentration of these proteins was similar to that in humans. Furthermore, the shape and size of the murine Hp 1 and Hp 2 proteins were similar to the human Hp 1 and Hp 2 proteins, respectively.

### Anesthesia

Mice were anesthetized by intraperitoneal injection of a mixture of xylazine (10 mg/kg [100 mg/ml Xylaject®, Phoenix Pharmaceutical, Inc., St. Joseph, Mo.]) and ketamine (50 mg/kg [100 mg/ml Ketaject®, Phoenix Pharmaceuticals, Inc., St. Joseph, Mo.]).

### Surgical Technique

The surgical protocol was modified from the procedure described by Lin et al. [A murine model of subarachnoid hemorrhage-induced cerebral vasospasm. J Neurosci Methods 123, 89-97 (2003).] Briefly, after induction of anesthesia, the dorsal neck from the inion to C2 as well as the right lower abdominal quadrant to the mid-thigh, were shaved with clippers. These areas were then prepared with 95% ethyl alcohol followed by povidone iodine solution. The animals were placed prone with their heads flexed approximately 300 below horizontal, and a midline incision was made from the inion to Cl. With the aid of the operating microscope (Zeiss Co., Oberkochen, Germany), the neck muscles were dissected off the occipital bone and retracted inferiorly until the atlanto-occipital membrane was exposed. The animals were then placed supine and an incision was made from the right anterior superior iliac spine to the groin region. The right femoral artery was exposed and 60 μl of autologous blood was withdrawn. An equivalent volume of normal saline solution (60 μl) was replaced intraperitoneally after blood removal. The animals were then placed prone and the atlanto-occipital membrane was re-exposed. The membrane was punctured using a 30-gauge needle directed 45° caudally, and, depending on the experimental group, either 60 μl of autologous blood or 60 μl of normal saline solution was slowly injected into the cisterna magna over a 2 minute span. The animals were then positioned head down for 30 minutes to confine the blood to the intracranial cisterns. The neck and leg tissues were re-approximated, and the incisions closed with staples.

### Activity Level Assessment

The mice were evaluated postoperatively for changes in neurological status, such as decreased activity level, paraparesis, anorexia, and impaired grooming. Buprenorphine (0.05 mg/kg [0.3 mg/ml, Abbott Laboratories, Abbott Park, Ill.]) was administered prophylactically every 12 hours for analgesia. Since peak vasospasm in mice occurs 24 hours after injection of blood into the cisterna magna, the activity level of the animals was recorded at 24 hours following surgical procedure. The activity level was assessed using a three point scale that evaluates their posture, grooming, and ambulation (Table 1).

TABLE I
Three-point scale used to assess the mouse's activity level at 24 hours
following induction of SAH. which corresponds to the time of peak
vasospasm in mice

| | 1 | 0 |
|---|---|---|
| Posture | Mouse is primarily in a | Mouse is primarily in an |
| | non-huddled position | arched back or huddled |
| | | position |
| Grooming | Fur is shiny and smooth | Fur appears oily and |
| | | ruffled |
| Ambulation | Mouse is alert, readily | Mouse is non-alert and |
| | ambulates around the | non-ambulatory |
| | cage, and is able to stand | |
| | on its hind legs | |

### Histology Preparation for Luminal Patency and Leukocyte Infiltration Analysis

Peak vasospasm in mice, as described above, occurs 24 hours following injection of blood into the cisterna magna. Therefore, mice were anesthetized at 24 hours post-SAH as described above for the SAH procedure. Perfusion-fixation was performed by making a midline incision from the sternal notch to the epigastrium, opening the right atrium, and cannulating the left ventricle with a 26-gauge butterfly needle. Perfusion was begun with normal saline solution at a flow rate of 5 ml/min for 10 minutes, followed by fixation with 4% paraformaldehyde (PFA) (Sigma 158127, St. Louis, Mo.) in 0.1 mol/L phosphate buffered saline (PBS) (pH 7.4) (Gibco 10010023, Gaithersburg, Md.) at the same flow rate using a fluid pump (Watson-Marlow 5058, Watson-Marlow Inc., Wilmington, Mass.). The brain was then harvested en bloc, and the brainstem with the basilar artery was removed from the rest of the brain and immersed in 4% PFA overnight at 4° C. After fixation, the tissue was placed in 30% sucrose solution (Sigma S0389) at 4° C. for 24 hours for cryoprotection. The tissue section was then frozen in Optimal Cutting Temperature (OCT) Compound (Tissue-Tek 83, Torrance, Calif.), and transverse sections (20 microns) were obtained with a microtome cryostat (Zeiss HM 5000M) at 60 micron intervals beginning at the basilar termination. Tissue slices were mounted on Superfrost Plus Slides (Fisher Scientific 12-550-15, Pittsburgh, Pa.) for either hematoxylin-eosin staining for luminal measurements or immunohistochemical staining for macrophage/neutrophil infiltration analysis.

### Lumen Patency Analysis

Hematoxylin-eosin stained histological sections of the basilar artery were digitized, the lumina were outlined at the transition zone between the internal elastic lamina and the tunica media, and the circumferences were measured using computerized analysis (MCID, Imaging Research, Inc., St. Catherines, Ontario, Canada). The circumferences were measured, instead of the area, to correct for vessel deformation and off-transverse sectioning.

### Leukocyte Infiltration Analysis

Mounted tissue sections were hydrated in PBS, and then incubated in 3% H₂O₂ for 5 minutes. The sections were then rinsed and incubated in a blocking solution consisting of 3% normal goat serum (S-1000, Vector Laboratories) diluted in PBS for 60 minutes. Following incubation, the sections were incubated with rat anti-mouse Ly-6G/6C primary antibody (BD 550291) diluted 1:25 in blocking solution in a humidified chamber for 60 minutes. This antibody identifies macrophages and neurtrophils, but not erythroid cells or cells of non-hematopoetic origin. The sections were then washed with PBS and incubated with biotin-conjugated anti-rat IgG2b secondary antibody (BD 550327) diluted 1:100 in blocking solution for an additional 60 minutes in a humidified chamber, followed by streptavidin-HRP (BD 550946) for 60 minutes. The sections were washed with PBS and then incubated in a DAB staining solution (BD 550880) for 8 minutes. The sections were then rinsed with tap water, counterstained with cresyl violet, and mounted with DPX mount (Sigma 44581) for visualization. The negative control was prepared by omitting the primary antibody. Each section was evaluated for the number of macrophages/neutrophils per high powered field (HPF).

### Statistical Analysis

For lumen patency analysis, the average artery circumference values for each of the animals were determined. These values were then converted into average area values by dividing the average circumference for each animal by 2π to determine the radii, and squaring these values and multiplying by π to determine the area. Mean cross-sectional areas were then expressed as percentage of lumen patency (% lumen patency) by dividing the mean vessel area of each animal by the mean area of the control group that did not undergo cisterna magna injection. The activity level was assessed using the three point scale detailed in Table 1. For macrophage/neutrophil infiltration analysis, all positive staining cells per representative high-powered field in a region adjacent to the adventitia of the basilar artery were counted. For each animal, three high powered-fields were counted.

To determine if there was a significant difference among the groups, mean vessel areas, activity level, and macrophage/neutrophil infiltration counts were compared using a one-way, non-parametric ANOVA (Kruskal-Wallis test), with the difference between each group determined by Student-Newman-Keuls' multiple comparison test using Sigmastat software 3.1 (Systat Software Inc., San Jose, Calif.). Values of p≦0.05 were considered significant.

The Hp 1-1 and Hp 2-2 cohorts were analyzed separately, with each genotype randomized into three experimental groups. Within each genotype, one group underwent injection of autologous blood into the cisterna magna (n=10), the second group underwent injection of normal saline solution (n=10), and the third group underwent no procedures (n=10). The brains of the animals were perfused, fixed, and harvested 24 hours following blood or saline injection. This time period corresponds to peak vasospasm development in mice.

### Example 1

### Lumen Patency Following SAH in Hp 1-1 and HP 2-2 Mice

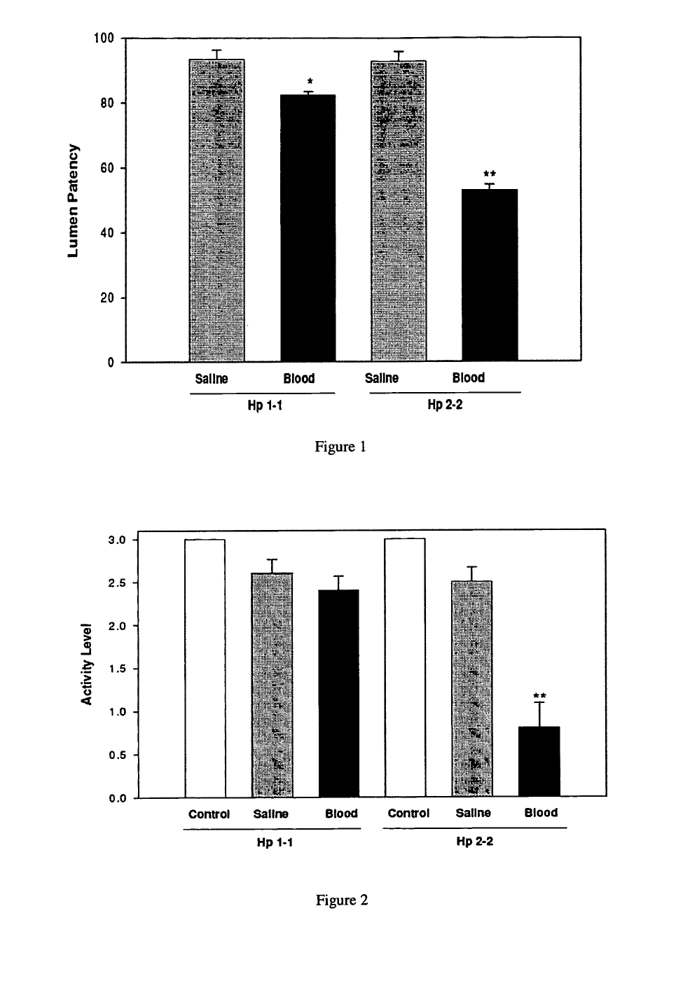

The percent lumen patency of the basilar artery was determined by dividing the mean vessel area of each animal by the mean area of the control group that did not undergo surgery. After SAH, the percent lumen patency (mean±SEM) was significantly reduced in blood-injected Hp 2-2 mice as compared to that of blood-injected Hp 1-1 mice (52.9±1.9% vs. 82.3±1.3%, p<0.001) (FIG. 1). Whereas the lumen patencies for Hp 1-1 control, saline, and blood-injected groups were 100±2.1%, 91.6±2.9%, and 82.3±1.3%, respectively, those of Hp 2-2 control, saline, and blood-injected groups were 100±3.8%, 92.8±3.0%, and 52.9±1.9%, respectively (FIG. 1). There were 10 animals per group.

### Example 2

### Activity Level Following SAH in Hp 1-1 and Hp 2-2 Mice

Activity levels were assessed 24 hours following surgery, according to the three-point scale described in Table 1. After SAH, the activity level (mean±SEM) was significantly reduced in Hp 2-2 mice as compared to that of Hp 1-1 mice (0.8±0.3 vs. 2.4±0.2, p<0.001) (FIG. 2). Whereas the activity levels for Hp 1-1 control, saline, and blood-injected groups were 3.0±0, 2.6±0.2, and 2.4±0.2, respectively, those of Hp 2-2 control, saline, and blood-injected groups were 3.0±0, 2.5±0.2, and 0.8±0.3, respectively (FIG. 2). There were 10 animals per group.

### Example 3

### Macrophage/Neutrophil Infiltration Following SAH in Hp 1-1 and Hp 2-2 Mice

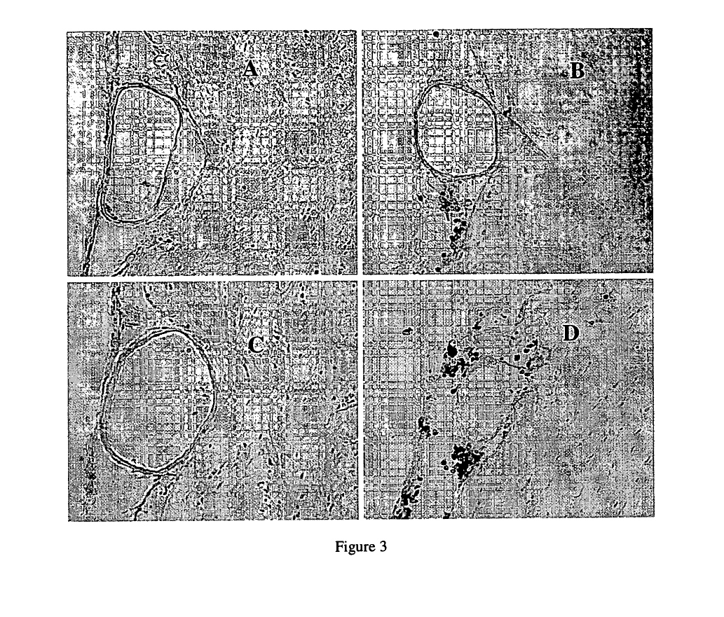

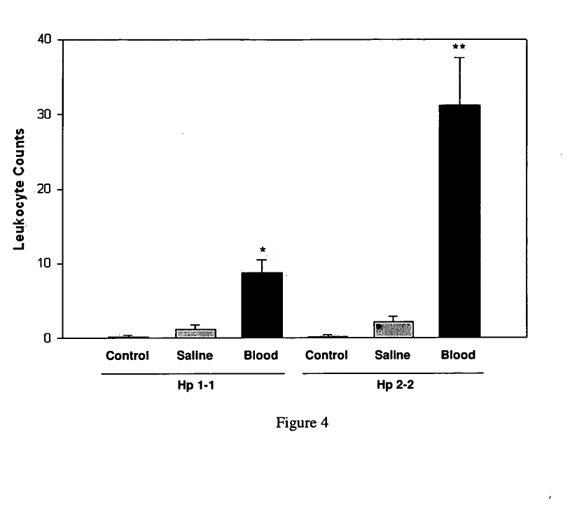

Macrophage/neutrophil infiltration was determined by counting the number of macrophages/neutrophils in the subarachnoid space per HPF in basilar artery sections (FIG. 3). After SAH, the number of macrophages/neutrophils (mean±SEM) in the subarachnoid space per HPF was significantly higher in Hp 2-2 mice as compared to Hp 1-1 mice (31.2±6.3 vs. 8.8±1.7, p=0.009) (FIG. 4). Whereas the number of macrophages/neutrophils in the subarachnoid space for Hp1-1 control, saline, and blood-injected groups was 0.2±0.2, 1.2±0.6, and 8.8±1.7, respectively, those of Hp 2-2 control, saline, and blood-injected groups was 0.2±0.3, 2.2±0.7, and 31.2±6.3, respectively (FIG. 4). There were 5 animals per group.

### Example 4

### Treatment of Vasospasm after SAH

The mouse model of SAH is carried out as described above in Lin et al., A murine model of subarachnoid hemorrhage-induced cerebral vasospasm. J Neurosci Methods 123, 89-97 (2003). Glutathione peroxidase mimetic is administered prior to or after the peak period for the occurrence of vasospasm, 24 hours after induction. Administration is found to improve activity scores.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of providing a prognosis for development of vasospasm in a subject, comprising the steps of: obtaining a biological sample from a subject following a hemorrhagic event; determining the haptoglobin (Hp) genotype in the biological sample, whereby a subject expressing a Hp-2-2 genotype has a high risk of developing vasospasm; and providing the prognosis based on the subject's haptoglobin genotype.

2. The method of claim 1, whereby the hemorrhagic event is a traumatic brain injury, a craniotomy for tumors, a meningitis, a subarachnoid hemorrhage (SAH), or their combination.

3. The method of claim 1, whereby said step of determining said haptoglobin genotype is effected by a signal amplification method, a direct detection method, a detection of at least one sequence change, an immunological method or a combination thereof.

4. The method of claim 3, whereby said signal amplification method amplifies a DNA molecule, an RNA molecule, or a combination thereof.

5. The method of claim 3, whereby said signal amplification method is PCR, LCR (LAR), Self-Sustained Synthetic Reaction (3SR/NASBA), Q-Beta (Qβ) Replicase reaction, or a combination thereof.

6. The method of claim 3, whereby said direct detection method is a cycling probe reaction (CPR), a branched DNA analysis, or a combination thereof.

7. The method of claim 3, whereby said detection of at least one sequence change employs a restriction fragment length polymorphism (RFLP analysis), an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF).

8. The method of claim 3, whereby step of determining said haptoglobin genotype is effected by an immunological detection method.

9. The method of claim 8, whereby said immunological detection method is a radio-immunoassay (RIA), an enzyme linked immunosorbent assay (ELISA), a western blot, an immunohistochemical analysis, or fluorescence activated cell sorting (FACS), or a combination thereof.

10. The method of claim 1, whereby the step of providing a prognosis for development of vasospasm as a result of hemorrhagic event, further comprises determining the importance of reducing oxidative stress in the subject.

11. The method of claim 1, whereby the subject is diabetic.

12. A system for providing a prognosis for development of vasospasm in a subject, comprising: a reagent, a packaging material; and instructions for determining the subject's haptoglobin genotype.

13. The system of claim 12, wherein a subject affected by a hemorrhagic event, expressing Hp-2-2 allele is at a high risk of developing vasospasm.

14. The system of claim 12, wherein the reagent enables a signal amplification method, a direct detection method, a detection of at least one sequence change, an immunological method or a combination thereof.

15. The system of claim 12, wherein said signal amplification method amplifies a DNA molecule, an RNA molecule, or a combination thereof.

16. The system of claim 12, wherein said signal amplification method is PCR, LCR (LAR), Self-Sustained Synthetic Reaction (3SR/NASBA), Q-Beta (Qβ) Replicase reaction, or a combination thereof.

17. The system of claim 12, wherein said direct detection method is a cycling probe reaction (CPR), a branched DNA analysis, or a combination thereof.

18. The system of claim 12, wherein said detection of at least one sequence change employs a restriction fragment length polymorphism (RFLP analysis), an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF).

19. The system of claim 12, wherein determining said haptoglobin genotype is effected by an immunological detection method.

20. The system of claim 19, wherein said immunological detection method is a radio-immunoassay (RIA), an enzyme linked immunosorbent assay (ELISA), a western blot, an immunohistochemical analysis, or fluorescence activated cell sorting (FACS), or a combination thereof.

21. The system of claim 12, further comprising reagents and instructions for determining the importance of reducing oxidative stress in the subject.

22. The system of claim 12, wherein the hemorrhagic event is a traumatic brain injury, a craniotomy for tumors, a meningitis, a subarachnoid hemorrhage (SAH), or their combination.

23. A method of treating a vasospasm, inhibiting or suppressing a vasospasm, or reducing symptoms associated with a vasospasm in a subject, comprising: contacting said subject, wherein the subject has suffered a hemorrhagic event with an effective amount of a composition comprising an antioxidant or its isomer, metabolite, and/or salt therefore, thereby reducing symptoms associated with vasospasm.

24. The method of claim 23, whereby said subject is diabetic.

25. The method of claim 23, whereby said antioxidant, an isomer, a functional derivative, a synthetic analog, is glutathione peroxidase mimetic represented by the compound of formula I:

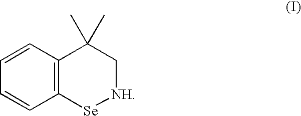

26. The method of claim 23, whereby said antioxidant or its isomer, metabolite, and/or salt therefore, is benzisoselen-azoline or -azine derivatives of glutathione peroxidase represented by the following general formula II: 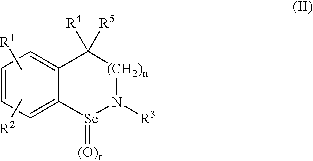
wherein R¹=R²=hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; (CH₂)ₘCOR⁸; —S(O)NR⁶R⁷; —SO₂NR⁶R⁷; —CO(CH₂)ₚCOR⁸; R⁹;
R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷=lower alkyl; aralkyl; substituted aralkyl; —(CH₂)ₘCOR⁸;
R⁸=lower alkyl; aralkyl; substituted aralkyl; aryl; substituted aryl; heteroaryl; substituted heteroaryl; hydroxy; lower alkoxy;
R⁹ is represented by any structure of the following formulae: 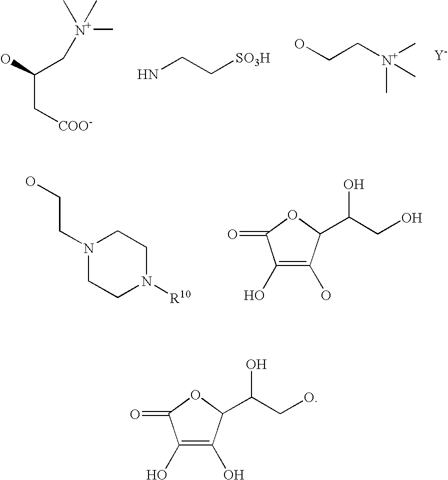
R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

27. The method of claim 23, whereby the antioxidant or its isomer, metabolite, and/or salt therefore is a glutathione peroxidase mimetic represented by the compound of formula III: 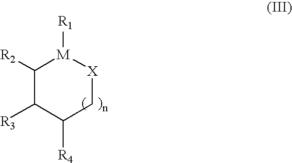
wherein,
the compound of formula I is a ring; and
X is O or NH
M is Se or Te
n is 0-2
R¹ is oxygen; and forms an oxo complex with M; or
R¹ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl;
wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is O, R₂ and R₃ form together with the organometallic ring a fused benzene ring, R₄ is hydrogen, then M is Se; or
if R₄ is an oxo group, and R₂ and R₃ form together with the organometallic ring a fused benzene ring, R₁ is oxygen, n is 0 and forms together with the metal a first 5 member ring, substituted by an oxo group a to R₁, and said ring is fused to a second benzene ring, then M is Te.

28. The method of claim 27, whereby the compound of formula III is represented by the compound of formula IV: 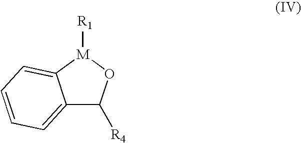
wherein, M, R₁ and R₄ are as described above.

29. The method of claim 27, whereby the compound of formula III is represented by the compound of formula V: 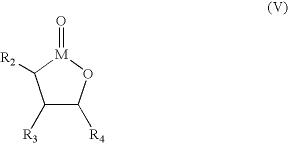
wherein, M, R₂, R₃ and R₄ are as described above.

30. The method of claim 27, whereby the compound of formula III is represented by the compound of formula VI: 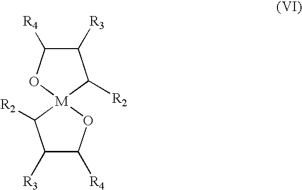
wherein, M, R₂, R₃ and R₄ are as described above.

31. The method of claim 27, whereby the compound of formula III is represented by the compound of formula VII: 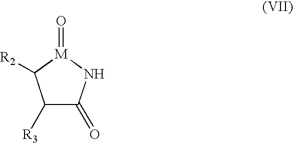
wherein, M, R₂, and R₃ are as described above.

32. The method of claim 27, whereby the compound of formula III is represented by the compound of formula VIII: 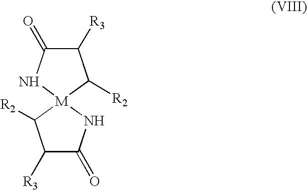
wherein, M, R₂, and R₃ are as described above.

33. The method of claim 27, whereby the compound of formula III is represented by the compounds:

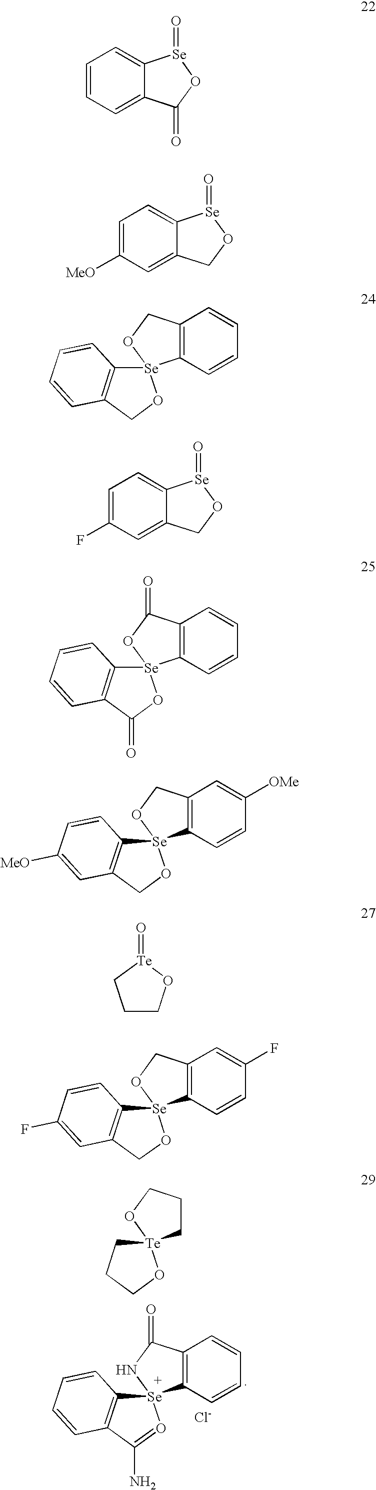

34. The method of claim 23, whereby the antioxidant or its isomer, metabolite, and/or salt therefore is a glutathione peroxidase mimetic represented by the compound of formula IX: 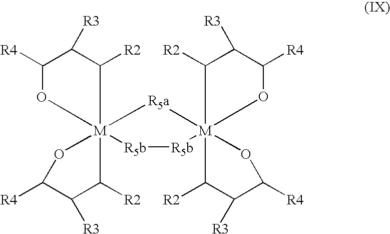
wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

35. The method of claim 34, whereby the compound of formula IX is represented by the compound of formula X:

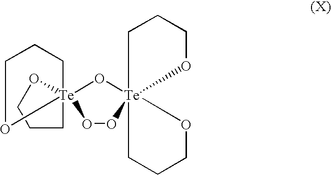

36. The method of claim 23, whereby the hemorrhagic event is a traumatic brain injury, a craniotomy for tumors, meningitis, a subarachnoid hemorrhage (SAH), angioplasty, or their combination.

37. The method of claim 23, preceded by determining the Hp phenotype in said subject.

38. A composition for treating a vasospasm in a subject wherein the subject suffered a hemorrhagic event, comprising: a therapeutically effective amount of a composition comprising an antioxidant or its isomer, metabolite, and/or salt therefore.

39. The composition of claim 38, wherein said antioxidant is a glutathione peroxidase mimetic, its isomer, functional derivative, or synthetic analog and their combination represented by any one of the compounds of formula I-X, or their combination.
